(19) United States
(12) Patent Application Publication
(10) Pub. No.: US 2007/0249827 A1
(43) Pub. Date: Oct. 25, 2007

(54) Meropenem Intermediatein in Crystalling Form

(75) Inventors: Peter Nadenik, Worgl (AT); Ole Storm, Kufstein (AT); Peter Kremminger, Kufstein (AT)

Correspondence Address: NOVARTIS;CORPORATE INTELLECTUAL PROPERTY, ONE HEALTH PLAZA 104/3, EAST HANOVER, NJ, 07936-1080, US

(73) Assignee: SANDOZ AG, Basle, CH (CH)

(21) Appl. No.: 11/569,611

(22) PCT Filed: Jun. 1, 2005
(86) PCT No.: PCT/EP05/05909

§ 371 Date: May 25, 2007

(30) Foreign Application Priority Data
Jun. 2, 2004 (AT) A944/2004

(51) Int. Cl. C07D 477/20

(52) U.S. Cl. 540/350

(57) ABSTRACT

The present invention relates to the compound (4R,5S,6S)-(p-nitrobenzyl) 3-[[(3S,5S)-1(-p-nitrobenzyloxycarbonyl)-5-(dimethylaminocarbonyl)-3-pyrrolidinyl]thio]-6-[(1R)-1-hydroxyethyl]-4-methyl-7-oxo-1-azabicyclo[3.2.0]hept-2-ene-2-carboxylate in crystalline form, as well as a process for the production of this crystalline compound in alkyl alkanoates, and its usage in a process for producing meropenem.

The present invention relates to (4R,5S,6S)-(p-nitrobenzyl) 3-[[(3S,5S)-1(-p-nitrobenzyloxycarbonyl)-5-(dimethylaminocarbonyl)-3-pyrrolidinyl]thio]-6-[(1R)-1-hydroxyethyl]-4-methyl-7-oxo-1-azabicyclo[3.2.0]hept-2-ene-2-carboxylate in crystalline form (hereinafter also referred to as “crystalline compound 1”) of formula 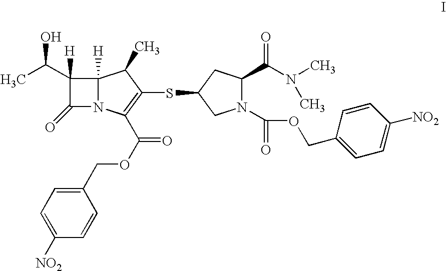
as well as processes for the production thereof.

The compound of formula I is an important known intermediate for the production of (4R,5S,6S)-3-[[(3S,5S)5-(dimethylaminocarbonyl)-3-pyrrolidinyl]thio]-6-[(1R)-1-hydroxyethyl]-4-methyl-7-oxo-1-azabicyclo[3.2.0]hept-2-ene-2-carboxylic acid (meropenem), a parenteral carbapenem having outstanding antibacterial properties. The compound of formula I in non-crystalline form, e.g. in amorphous form, in the form of an oil or in the form of a foamy or vitreous solid, is known. Compared with crystalline forms of a compound, amorphous forms generally have considerably poorer material properties, such as stability, isolating properties or purity. In the multi-stage synthesis of a product which is to be obtained in sufficiently high purity, it can therefore be an enormous advantage to use crystalline forms of intermediate compounds instead of amorphous forms. A crystallisation step normally leads to a high purifying effect, and in addition, crystalline materials have substantially better stability than amorphous products.

The present invention is therefore based on the problem of providing a crystalline intermediate compound, which has the above-mentioned advantages and can be used in preparation processes for meropenem.

This problem is solved according to the invention by the compound of formula I in crystalline form. It has been found that, under certain conditions, the compound of formula I can be obtained in crystalline form.

Therefore, one aspect of this invention relates to the compound of formula I in crystalline form.

The physico-chemical properties of the compound of formula I in crystalline form may be described as follows:
1) External appearance of the crystals

bacilli or short needles
2) Melting point

156-159° C.
3) X-ray powder diffractogram

The compound of formula I in crystalline form shows characteristic bands at the 2-theta diffraction angles of about 5.1, 12.8, 15.5, 17.6, 18.4, 21.1, 21.5 and 23.4.

The compound of formula 1 in crystalline may also be described by the x-ray powder diffractogram as shown in the following Table 1 and as depicted in FIG. 1.

Table 1: X-ray powder diffractrogram of the compound of formula I in crystalline form; apparatus used: powder diffractometer AXS-BRUKER D-8, CuK-α radiation, Bragg-Brentano-Optics, scintillation counter, 40 kV, 40 mA, 0.01° steps, time 2 seconds, cut-off: 40°, standard sample carrier; evaluation was carried out using the software DiffracPlus™.

| angle | d value | intensity |
|---|---|---|
| 2-Theta ° | Angstrom | % |
| 5.091 | 17.34318 | 18.9 |
| 12.815 | 6.90257 | 32.3 |
| 13.181 | 6.71159 | 20.2 |
| 15.010 | 5.89754 | 22.7 |
| 15.480 | 5.71953 | 29.3 |
| 15.848 | 5.58766 | 27.5 |
| 16.348 | 5.41781 | 19.7 |
| 17.608 | 5.03292 | 79.6 |
| 18.367 | 4.82644 | 100.0 |
| 19.758 | 4.48985 | 22.8 |
| 20.316 | 4.36776 | 20.1 |
| 20.602 | 4.30770 | 18.3 |
| 21.133 | 4.20055 | 36.7 |
| 21.477 | 4.13404 | 37.5 |
| 22.120 | 4.01542 | 28.0 |
| 22.404 | 3.96519 | 31.0 |
| 23.411 | 3.79678 | 33.6 |
| 25.872 | 3.44089 | 22.5 |
| 26.605 | 3.34785 | 24.4 |
| 27.545 | 3.23564 | 31.7 |

[image]

In a further aspect, the present invention relates to a process for the production of the compound of formula I in crystalline form.

The compound of formula I in crystalline form may be obtained whereby the compound of formula I in non-crystalline form is dissolved in an alkyl alkanoate, optionally also in a mixture with another organic solvent which is readily miscible with water. The solution of the compound of formula I may also be produced in situ by a coupling reaction between (2S,4S)-2-dimethylaminocarbonyl-4-mercapto-1-(p-nitrobenzyloxycarbonyl)-pyrrolidine or the salts thereof and a p-nitrobenzyl-(1R,5R,6S)-6-[(1R)-1-hydroxyethyl]-1-methylcarbapen-2-em-3-carboxylic acid ester which is activated at position 2 of the carbapenem frame, e.g. p-nitrobenzyl-(1R,5R,6S)-6-[(1R)-1-hydroxyethyl]-2-[(diphenylphosphono)oxy]-1-methylcarbapen-2-em-3-carboxylic acid ester, without intermediate isolation. Such coupling reactions are described, for example, in EP-A-126587.

If necessary, extraction with water follows, possibly even several times in succession, whereby the proportion of solvent miscible with water in the ethyl acetate phase continuously decreases.

The compound of formula I in crystalline form crystallises out of this alkyl alkanoate solution when the conditions necessary for crystallisation have been set. To set the conditions necessary for crystallisation, measures which allow crystallisation to set in and are known to a person skilled in the art, are appropriate, for example at least partly removing the solvent, such as by evaporating the solvent, optionally at reduced pressure, and/or by cooling the solvent, and/or by continually stirring. In addition, an anti-solvent may be added in order to complete crystallisation. A polar hydrocarbons are especially suitable anti-solvents, e.g. aromatic hydrocarbons such as benzene, toluene, xylenes or alkanes, such as (C₆₋₁₂)-alkanes, e.g. (C₆₋₈)-alkanes, or cycloalkanes, such as (C₅-C₈)cycloalkanes. It is especially preferable to use heptane or cyclohexane, especially cyclohexane, as anti-solvents. Alkyl alkanoates which are suitable as solvents are, for example, (C₁₋₆)alkyl esters of (C₂₋₄)-alkanoic acids. (C₁₋₆)alkyl esters of C₂-alkanoic acid (acetic acid) are preferred, especially (C₂₋₄)alkyl esters of acetic acid, such as ethyl acetate, isopropyl acetate, n-propyl acetate, n-butyl acetate, isobutyl acetate, sec.-butyl acetate or tert.-butyl acetate. Ethyl acetate, isopropyl acetate or n-butyl acetate are preferred in particular, especially ethyl acetate.

The crystalline compound of formula I which has crystallised out is isolated in the usual manner, e.g. filtered, washed and dried in a vacuum. The purity of the crystalline compound of formula I, which is obtained by the described process, is typically more than 98.5% (expressed as HPLC area percent), normally even more than 99.0% (HPLC area percent).

The yields are excellent and are normally at least 90% of theory, optionally up to 95% of theory.

A further aspect of this invention relates to the use of the compound of formula I in crystalline form, e.g. characterised by the powder diffractogram according to FIG. 1, especially as an intermediate compound, for the production of meropenem.

The compound of formula I in crystalline form may be isolated very easily, and has excellent purity and stability. The compound of formula I in crystalline form is therefore very suitable as an intermediate, particularly also on an industrial scale, in the process for producing meropenem. In addition, usage of the compound of formula I in crystalline form has the advantage that other purification steps, such as chromatography, can be dispensed with, since the purity of the compound of formula I in crystalline form is completely sufficient for the production of meropenem.

TABLE 2
Stability comparisons between compound of formula I in
crystalline form and amorphous form

| | Compound of formula I | Compound of formula I in |
|---|---|---|
| | in crystalline form | amorphous form |
| starting value: | 99.4% by weight | 92.4% by weight |
| sum of all | <0.3% | 2.5% |
| by-products | | |
| 72 h at70° C. | 99.1% by weight | 90.8% by weight |
| sum of all | <0.3% | sum of all by-products 5.7% |
| by-products | | |

These points clearly indicate that the use of the compound of formula I in crystalline form brings with it considerable advantages over the usage of the previously described amorphous forms of the compound of formula I, which may be reacted and employed especially in the production of meropenem.

Therefore, in a further aspect, the present invention relates to a process for the production of meropenem, which is characterised in that it comprises a step of isolating the compound of formula I in crystalline form, which is then further reacted to meropenem, especially by cleaving the p-nitrobenzyl or p-nitrobenzyloxycarbonyl protecting groups from the carboxyl group in position 2 of the carbapenem frame, as well as from the nitrogen atom of the pyrrolidine ring of the side chain. Cleavage may take place analogously to, e.g. exactly as in known processes for the production of meropenem, for example by hydrogenation as described in EP-A-126587.

The following examples are intended to illustrate the present invention and should in no way limit its scope. All temperatures are indicated in ° C., and all purities in HPLC area percentages.

### EXAMPLE 1

### Crystalline (4R,5S,6S)-(p-nitrobenzyl) 3-[[(3S,5S)-1(-p-nitrobenzyloxycarbonyl)-5-(dimethylaminocarbonyl)-3-pyrrolidinyl]thiol]-6-[(1R)-1-hydroxyethyl]-4-methyl-7-oxo-1-azabicyclo[3.2.0]hept-2-ene-2-carboxylate

p-nitrobenzyl-(1R,5R,6S)-6-[(1R)-1-hydroxyethyl]-2-[(diphenylphosphono)oxy]-1-methylcarbapen-2-em-3-carboxylic acid ester (23.5 g, 39.5 mmols) and (2S,4S)-2-dimethylaminocarbonyl-4-mercapto-1-(p-nitrobenzyloxycarbonyl)-pyrrolidine (14.8 g, 41.9 mmols) are dissolved in 100 ml of dimethylacetamide and cooled to −10° C. Over the course of 15 mins, 22 ml of diisopropylethyl amine are added dropwise in such a way that the temperature does not exceed −7° C. The mixture is stirred for 1 hour at −10° C. Then, 300 ml of cold ethyl acetate are added over 30 mins at a temperature of <−7° C. and finally 200 ml of ice water are added at <−5° C. over 30 mins.

The aqueous phase is separated and extracted with 150 ml of ethyl acetate. The combined organic phases are extracted twice, each time with a cold mixture of 80 ml of 6% aqueous NaCl solution and 20 ml of 2N hydrochloric acid, and once with 100 ml of phosphate buffer solution pH 7.0.

The organic phase is separated, filtered and the filter washed with 10 ml of ethyl acetate. The filtrate is concentrated to 90 g at 40° C. and made up to 110 g with ethyl acetate and stirred for 72 h at 20° C. The product crystallises. In order to complete crystallisation, 35 ml of heptane are added dropwise and the crystal suspension is stirred for 30 mins. The crystalline product is isolated by filtration, washed twice, each time with 50 ml of heptane, and dried for 16 h at 40° C. in a vacuum.

Weighed product: 25.45 g (36.5 mmols)

purity (HPLC): 98.6%

Melting point: 156-159° C.

IR (Golden gate) cm⁻¹: 1762.6, 1702.6, 1638.4, 1519.3, 1444.0, 1402.4, 1341.2, 1317.0, 1273.4, 1205.6, 1179.2, 1138.2, 1111.9, 1045.4, 1017.1, 985.1, 860.0, 841.1, 767.1, 739.4 ¹H-NMR (d₆-DMSO) δ 8.24 (m, 4H), 7.73 (d, J=9.00, 2H), 7.66&7.54 (d, J=9.0, 2H), 5.47&5.30 (ABq, 2H,J=14.1 Hz), 5.25-5.04 (m,3H), 4.80 (m,1H), 4.26 (m,1H), 4.15 (m,1H), 3.99 (m,1H), 3.86 (m,1H), 3.61 (m,1H), 3.29 (m,1H), 3.20 (m,1H), 3.03&2.97 (2s,3H), 2.87 (m,1H), 2.83&2.83 (2s,3H), 1.63 (m,1H), 1.17 (2t, 6H,J=6.0 Hz)

### EXAMPLE 2

### Crystalline (4R,5S,6S)-(p-nitrobenzyl) 3-[[(3S,5S)-1(-p-nitrobenzyloxycarbonyl)-5-(dimethylaminocarbonyl)-3-pyrrolidinyl]thiol]-6-[(1R)-1-hydroxyethyl]-4-methyl-7-oxo-1-azabicyclo[3.2.0]hept-2-ene-2-carboxylate

2.5 g of amorphous (4R,5S,6S)-(p-nitrobenzyl) 3-[[(3S,5S)-1(-p-nitrobenzyloxycarbonyl)-5-(dimethylaminocarbonyl)-3-pyrrolidinyl]thio]-6-[(1R)-1-hydroxyethyl]-4-methyl-7-oxo-1-azabicyclo[3.2.0]hept-2-ene-2-carboxylate (content ca. 90%, HPLC purity: 97.7 area %) are dissolved in 42 ml of ethyl acetate. The amorphous material rapidly goes into solution. The solution is stirred at 20° C., and after about 10 mins, crystallisation sets in. After 1.5 h, 3.5 ml of cyclohexane are added in order to complete crystallisation. The crystal suspension is stirred for 2.5 h.

The crystalline product is isolated by filtration, washed with 5ml of cyclohexane, and dried for 16 h at 40° C. in a vacuum.

Weighed product: 1.96 g

HPLC purity: 99.8%

Melting point: 159-161° C.

¹H-NMR (d₆-DMSO, 300 MHz): identical to above

### EXAMPLE 3

### Further Processing of Crystalline Compound of Formula I Into Meropenem

1.0 g of the compound obtained in example 1 is dissolved in 60 ml of tetrahydrofuran and 90 ml of water, mixed with 1.0 g of 10% palladium on activated carbon and hydrogenated at atmospheric pressure for 3 h. Subsequently, 50 ml of ethyl acetate are added. Filtration takes place and the organic phase is separated. The aqueous phase is washed once with ethyl acetate and carefully concentrated to 5 ml. 30 ml of tetrahydrofuran are added dropwise to the aqueous solution, which is then seeded and allowed to crystallise at 0° C. The crystalline precipitate of meropenem is filtered off, washed with tetrahydrofuran and dried in a vacuum.

Weighed product: 0.48 g

¹H-nmr (D₂O)?δ 1.21 (d,3H,J=7.1 Hz), 1.29 (d, 3H,J=6.3 Hz), 1.97 (m,1H), 2.99 (s,3H), 3.06 (s,3H), 3.09 (m,1H), 3.38 (m,1H), 3.46 (m,2H), 3.77 (dd,1H,J=11.9&6.2 Hz), 4.05 (m,1H), 4.23 (m,2H), 4.82 (1H, signal is partly under D₂O signal)

1. A compound of formula 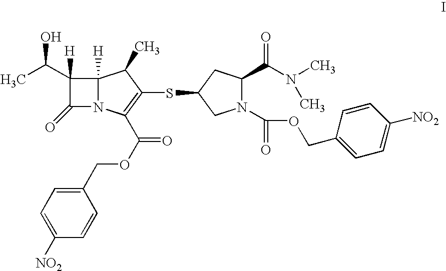
in crystalline form.

2. The compound according to claim 1 characterized by powder x-ray diffraction peaks at the 2-theta diffraction angles of about 5.1, 12.8, 15.5, 17.6, 18.4, 21.1, 21.5 and 23.4.

3. The compound according to claim 1, which has the following characteristic X-ray diffraction diagram:

| angle | d value | intensity |
|---|---|---|
| [2-Theta °] | [Ångstrom] | [%] |
| 5.091 | 17.34318 | 18.9 |
| 12.815 | 6.90257 | 32.3 |
| 13.181 | 6.71159 | 20.2 |
| 15.010 | 5.89754 | 22.7 |
| 15.480 | 5.71953 | 29.3 |
| 15.848 | 5.58766 | 27.5 |
| 16.348 | 5.41781 | 19.7 |
| 17.608 | 5.03292 | 79.6 |
| 18.367 | 4.82644 | 100.0 |
| 19.758 | 4.48985 | 22.8 |
| 20.316 | 4.36776 | 20.1 |
| 20.602 | 4.30770 | 18.3 |
| 21.133 | 4.20055 | 36.7 |
| 21.477 | 4.13404 | 37.5 |
| 22.120 | 4.01542 | 28.0 |
| 22.404 | 3.96519 | 31.0 |
| 23.411 | 3.79678 | 33.6 |
| 25.872 | 3.44089 | 22.5 |
| 26.605 | 3.34785 | 24.4 |
| 27.545 | 3.23564 | 31.7 |

4. A process for the production of the compound of formula I as defined in claim 1 in crystalline form, comprising the step in which the compound of formula I in non-crystalline form is dissolved in an alkyl alkanoate and crystallisation of the compound of formula I is carried out in order to obtain the compound of formula I in crystalline form.

5. The process according to claim 4, whereby the alkyl alkanoate is a (C₁₋₆)alkylester of acetic acid.

6. The process according to claim 5, whereby the alkyl alkanoate is isopropyl acetate, n-butyl acetate or ethyl acetate.

7. The process according to claim 4, whereby an anti-solvent is added.

8. The process according to claim 7, whereby the anti-solvent is an a polar hydrocarbon.

9. A method of using the crystalline compound of formula I as defined in claim 1 in a process for producing meropenem.

10. A process for the production of meropenem comprising the isolation of the compound of formula I, as defined in claim 1, in crystalline form.

11. The process according to claim 10, in which the p-nitrobenzyl and/or p-nitrobenzyloxycarbonyl protecting groups of the compound of formula I, which is isolated in crystalline form, are removed in a further step.
